(19) United States
(12) Patent Application Publication
(10) Pub. No.: US 2026/0248418 A1
(43) Pub. Date: Aug. 27, 2026

(54) FULL-FABRIC MICROFLUIDIC SWEAT COLLECTING AND SENSING SYSTEM AND PREPARATION METHOD THEREOF

(71) Applicant: The Hong Kong Polytechnic University, Hong Kong (CN)

(72) Inventors: Dahua SHOU, Hong Kong (CN); Jinhao XU, Hong Kong (CN); Xungai WANG, Hong Kong (CN)

(21) Appl. No.: 19/531,779

(22) Filed: Feb. 6, 2026

Related U.S. Application Data
(60) Provisional application No. 63/763,329, filed on Feb. 26, 2025.

(30) Foreign Application Priority Data
Aug. 19, 2025 (CN) 202511159880.1

(51) Int. Cl. A61B 5/145; A61B 5/1477

(52) U.S. Cl. CPC A61B 5/14517; A61B 5/14525; A61B 5/14532; A61B 5/14546; A61B 5/1477

(57) ABSTRACT

A microfluidic sweat collecting and sensing system is provided. The system has a bottom layer close to a skin of a wearer, a top layer remote from the skin, and a separation layer between the bottom and top layers. The separation layer is made of a hot-melt material. The top layer, the bottom layer and the separation layer are bonded together by hot pressing, so that a gap is formed between the top layer and the bottom layer. The top and bottom layers are provided with water channels respectively. Fabrics forming the bottom and top layers are hydrophilic in parts corresponding to the water channels and hydrophobic in remaining parts.

### CROSS-REFERENCE TO RELATED APPLICATIONS

This application claims priority to, and the benefit of, U.S. Provisional Patent Application No. 63/763,329 filed on Feb. 26, 2025, and China Invention patent application No. 202511159880.1 filed on Aug. 19, 2025, the disclosures of both of which are incorporated by reference herein in their entirety.

### FIELD OF THE DISCLOSURE

The present invention relates to creation of a full-fabric microfluidic system for real-time sweat collection and biomarker monitoring.

### BACKGROUND

Existing microfluidic sweat sensors have drawbacks in terms of wear resistance, air permeability and cost effectiveness. Specifically, existing products of the existing microfluidic sweat sensors have the following limitations.

### 1. Insufficient Collection of Fresh Sweat, Leading to Incapability of Accurate Sensing

A real-time sensing performance of a sweat sensor in an existing wearable system depends, to a great extent, on a composition of sweat of a human body, and a change in the composition of the sweat seriously affects stability and accuracy of the sensor in sweat sensing. After the human body sweats, sweat dissipation depends mainly on evaporation. On one hand, new sweat is excreted to a skin surface, and on the other hand, excreted sweat constantly evaporates. When an evaporation rate is lower than a perspiration rate, the new sweat excreted to the skin surface may be mixed with old sweat. It may affect a concentration of a biomarker in the mixed sweat. Thus, in the existing wearable system, such as a fabric sensor, a sensor reading gives a moving average of distribution of an analyte in the sweat rather than a real-time precise measurement value. It leads to challenges in precisely reflecting an individual health condition.

For an existing measurement technology, the human body usually needs to profusely sweat making an adequate contact between the sweat and an electrode. However, the accumulated sweat can affect long-term sensing accuracy and fidelity of the sensor. Furthermore, people do not always sweat profusely unless under a strenuous exercise or an extremely hot climate. A current miniaturized electronic sensor that adheres to the skin of the human body can reduce sweat leakage, but a limited collection region below a microfluidic channel cannot provide a sufficient volume of sweat for precision sensing. Therefore, there is a need for a new sweat collecting and sensing system to quickly obtain sufficient fresh sweat under normal sweating of the human body.

### 2. Poor User Experience and Discomfort

In recent years, new technologies, including microfluidic systems, electrochemical sensors, microchips and wireless signal emitters, provide important foundations for sensing the sweat on the skin of the human body, but cause discomfort to a wearer. It severely hampers acceptance and popularization of wearable sensors in the industry and the market. A patch made of a plastic film and a silicone pad and pasted on the body has poor air permeability for the skin of the human body. It may cause inflammation of the skin to generate symptoms, such as redness, fever, itching and swelling. Fabric-based microfluidic sweat sensors have enhanced tactile properties and air permeability, especially under a dry condition. In a profuse sweating case, however, the sensors become saturated and heavy because a speed of sweat dissipation based on passive wicking and evaporation is not high enough. In addition, profuse sweating may cause skin damage and heat stress, and pores of a fabric permeated by the sweat reduce the air permeability and moisture permeability. Moisture and adhesion on the skin not only affect comfort and limit body movement, but also can lead to abnormal sweating and reduced sensor sensing precision.

### 3. Lack of Durability and Washability

Wearable sweat sensors with high durability and washability are highly desirable, but high durability and washability are difficult to achieve concurrently. Existing integrated wearable electronic sensors are typically compact, but long-term performances thereof, including safety and tensile strength for washing with clothing, still require comprehensive evaluation. The fabric microfluidic sweat sensors achieve a better balance between treatment and mechanical performances, but stable incorporation of electrochemical sensing materials on the fabric and fiber electrodes still needs to be further improved.

### SUMMARY

The present invention provides a full-fabric microfluidic system for addressing existing technical problems in the art. The system integrates a sensing function, reduces discomfort of a wearer, and optimizes collection, movement and dissipation of sweat, thereby improving accuracy and wearability. Specifically, the present invention provides rapid collection of fresh sweat and accurate sweat sensing as well as achieves great comfort and durability. The system controls transport of the sweat from the skin to the microfluidic system (normal transport) and then directs the sweat to a sensing region (transverse transport). The system according to the present invention can operate in two modes: a fully passive mode and an active-passive combined mode. In the fully passive mode, sweat transport relies on a humidity gradient, and the sweat is directed to move in a plane (transverse transport) through a specific geometric structure. In the active-passive combined mode, normal transport driving is performed by an electroosmotic force, and a geometric shape is used to guide in-plane movement (transverse transport). An electrochemical sensing yarn is wrapped in a skin-friendly fiber sewn in a sweat collection region, so that a treatment performance is not affected. An active layer of the sensing yarn includes glucose oxidase and an ion-selective membrane, and is sufficiently protected to ensure the high durability and washability. An ultra-light and detachable wireless signal transmission unit is arranged in a small pocket of the fabric sensor. The wearable sweat sensor according to the present invention has a higher sensing precision, a higher wear resistance and a greater durability. The system according to the present invention is applicable to sports T-shirts, head bands, wrist bands and other garments. In addition, the system may be suitable for detecting various biological fluids, including wound exudate, blood, tears, and urine, so that the system is suitable for use in wound dressings, eye patches, diapers and similar applications.

### BRIEF DESCRIPTION OF THE DRAWINGS

The present invention is described in further detail below with reference to the following drawings and embodiments. In the drawings:

FIG. 1A is a schematic diagram of a cross section of a microfluidic system in a fully passive mode according to some embodiments of the present invention;

FIG. 1B is a schematic diagram of the cross section of the microfluidic system in an active-passive combined mode according to some embodiments of the present invention, with a circuit schematically representing an electroosmotic flow apparatus;

FIG. 1C shows an exploded view of the electroosmotic flow apparatus of FIG. 1B in some embodiments;

FIG. 2A is a plan view of a bottom layer of the microfluidic system according to some embodiments of the present invention;

FIG. 2B is a plan view of a top layer of the microfluidic system according to some embodiments of the present invention;

FIGS. 2C and 2D are schematic diagrams of water channels in the bottom and top layers shown in FIGS. 2C and 2B, respectively;

FIG. 2E shows a schematic diagram of the water channel of the bottom layer with a wicking assembly according to some embodiments of the present invention;

FIG. 3A shows a perspective view of the bottom layer cut to form the water channel according to some embodiments of the present invention;

FIG. 3B shows a schematic diagram of normal flowing and transverse flowing of sweat secreted from the skin according to some embodiments of the present invention;

FIG. 4A shows a photograph of a sweat flowing mode on the water channel in a structure of the microfluidic system according to the present invention when a skin sweating simulator continuously supplies sweat at a flow rate of 2 μL/min/cm²;

FIG. 4B is an image of sweat accumulation in a sweat sensing region of a full-fabric microfluidic system with the wicking assembly;

FIG. 5A is a schematic diagram of functions of the electroosmotic flow apparatus according to some embodiments of the present invention;

FIG. 5B is an effect-showing view of normal sweat transport produced after the electroosmotic flow apparatus is switched off and on according to some embodiments of the present invention;

FIG. 5C shows a plan view of a large-scale electroosmotic flow apparatus according to some embodiments of the present invention;

FIG. 6 schematically shows a process for preparing an electrochemical sensing yarn according to some embodiments of the present invention;

FIG. 7A is a schematic diagram of preparation of a protective layer of the sensing yarn according to some embodiments of the present invention;

FIG. 7B is a design flow chart of a printed circuit board according to some embodiments of the present invention;

FIG. 8A is a graph of cyclic voltammetry measurement using a ferricyanide redox probe;

FIG. 8B is a bar graph of active surface areas of two yarn electrodes;

FIG. 8C is an impedance spectrum of the two yarn electrodes;

FIG. 9A shows a calibration graph of a K+ sensing electrode;

FIG. 9B shows a calibration graph of a glucose sensing electrode;

FIG. 10A shows a view of an anti-interference performance of the K+ yarn electrode;

FIG. 10B shows a graph of an anti-interference performance of the glucose yarn electrode;

FIG. 11A shows a graph of internal and external K+ signal voltage output stability with/without a microfluidic sweat collection system; and

FIG. 11B shows a graph of stability of current signal output for glucose with/without the microfluidic sweat collection system.

### DETAILED DESCRIPTION

The present invention will be described in further detail below with reference to the accompanying drawings and embodiments in order to make the objects, technical solutions, and advantages of the present invention more clear. It should be understood that the specific embodiments described herein are only for explaining the present invention, and not intended to limit the present invention.

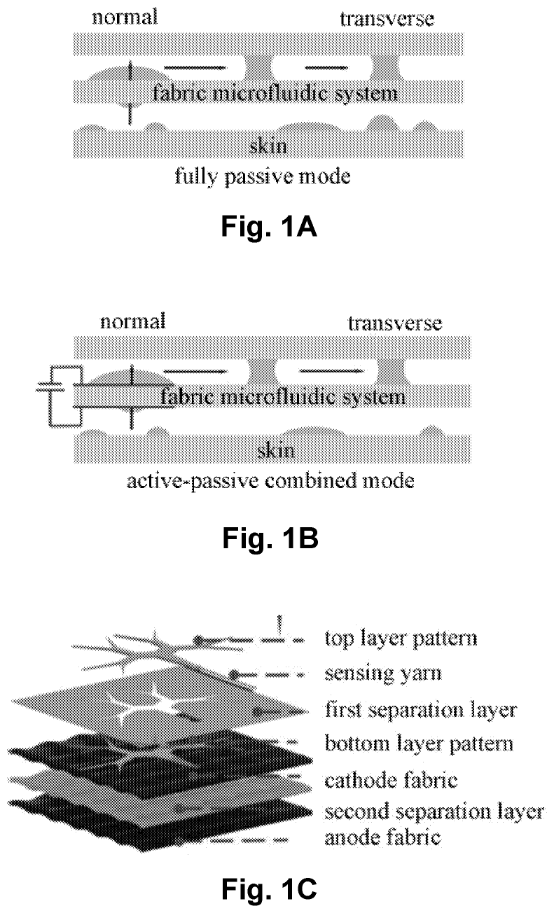

The present invention provides a full-fabric microfluidic system which includes a sweat collection assembly and a sensing assembly. The sweat collection assembly operates in two modes: a fully passive mode and an active-passive combined mode (FIGS. 1A and 1B). FIG. 1A is a schematic diagram of a partial cross section of a substrate of the microfluidic system in the fully passive mode according to some embodiments of the present invention. The microfluidic system shown in the drawing has a root branch design with a double-layer structure. The double-layer structure as shown includes a lower layer (i.e. a bottom layer) close to the skin, an upper layer (i.e. a top layer) remote from the skin, and a separation layer between the lower layer and the upper layer. Sweat secreted from the skin is first absorbed by the lower layer of the microfluidic system, and then diffused to the upper layer. The transport perpendicular to the microfluidic system or perpendicular to the skin is referred to herein as normal transport. Correspondingly, the sweat can also be transported along the microfluidic system or the skin, which is referred to herein as in-plane or transverse transport.

FIG. 1B is a schematic diagram of the partial cross section of the microfluidic system in the active-passive combined mode according to some embodiments of the present invention. Unlike FIG. 1A, an electroosmotic flow apparatus (described in detail below) is added to the bottom layer (lower layer) of the fully passive microfluidic system to facilitate transfer of the sweat from the skin into a microfluid.

In the fully passive mode (see FIG. 1A), the transport of the sweat secreted from the skin is entirely dependent on a humidity gradient, the sweat is diffused from a place with high humidity to a place with low humidity, and the transport includes the normal transport and the transverse (in-plane) transport. In the active-passive combined mode (see FIG. 1B), the electroosmotic flow apparatus facilitates the normal transport of the sweat. The electroosmotic flow apparatus is connected to the bottom layer (lower layer) of the microfluidic system, and transfers the sweat from the skin into the microfluidic system. Thus, a mechanism of the in-plane sweat transport is the same as that in the fully passive mode. In the sensing assembly, a yarn electrode is embroidered on a fabric and connected to a thin and flexible PCB for real-time wireless signal transmission.

FIG. 1C shows an exploded view of the electroosmotic flow apparatus as shown in FIG. 1B in accordance with some embodiments of the present invention. As can be seen in FIG. 1C, the electroosmotic flow apparatus includes a cathode fabric layer, an anode fabric layer and a separation layer (also referred to as a second separation layer) located therebetween. The cathode fabric layer and the anode fabric layer may be electrically connected to a cathode and an anode of a power source (not shown) respectively to generate an electric field therebetween to facilitate flowing of the sweat from the anode fabric layer to the cathode fabric layer. In addition, FIG. 1C also shows another separation layer (also referred to as a first separation layer) between the cathode fabric layer and the top layer of the sweat collecting and sensing system, a part of a material of the first separation layer is cut away by laser to form a pattern, and the pattern includes a sweat collection region and a sensing region (described in detail below). In addition, FIG. 1C also shows a pattern part of the top layer, the pattern part corresponds to a bottom layer pattern of the cathode, and the rest of the top layer is not shown for the sake of clarity. The top layer pattern corresponds to a pattern of the sweat collection region of the first separation layer and is connected with a sensing yarn.

Preparation of the microfluidic system according to the present invention is described in detail below.

### 1. Fully Passive Mode Microfluidic System

### Preparation of Substrate of Fully Passive Mode Microfluidic System

According to some embodiments of the present invention, the fully passive mode microfluidic system is prepared as follows. Before use, the fabric is washed with distilled water to remove impurities, foreign materials, or the like, on a surface of the fabric, and then dried in an oven. The fabric described herein may be a commercially available fabric having hydrophilicity, such as a cotton fabric. Aluminum oxide (Al₂O₃) nanoparticles and titanium butoxide (TT) are dispersed in deionized water at concentrations of 18 g/L and 12 g/L respectively to form Al₂O₃ hydrosol, and the hydrosol is then subjected to ultrasonic treatment for 30 minutes. Subsequently, the washed fabric is immersed into the obtained hydrosol at 40° C. and held for a short period of time (e.g., 15 to 25 minutes, preferably 20 minutes, or any time deemed appropriate by one skilled in the art), and then, the fabric is taken out. In a preferred embodiment, in order to ensure removal of the nanoparticles attached to the fabric, the fabric is rinsed twice with deionized water. Finally, the fabric is dried by air at an ambient temperature.

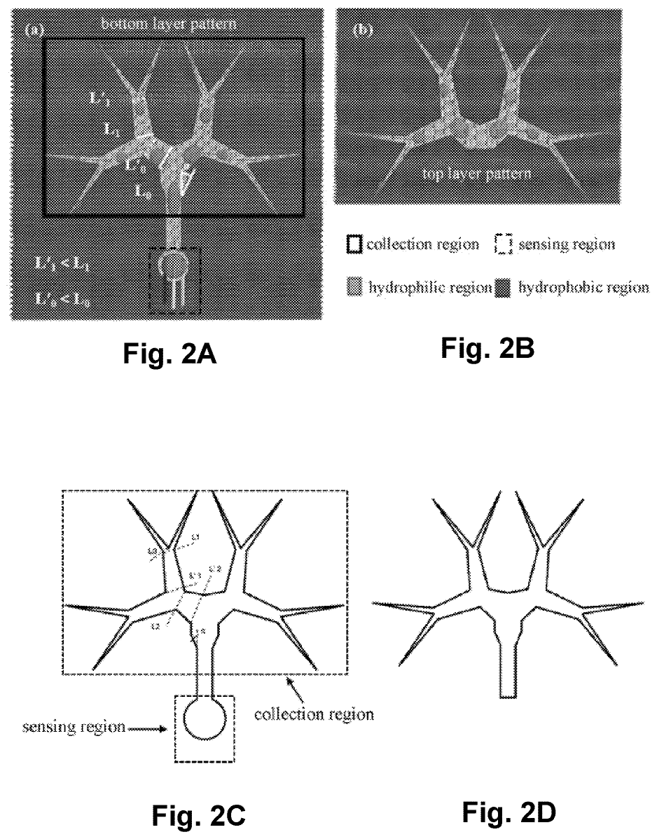

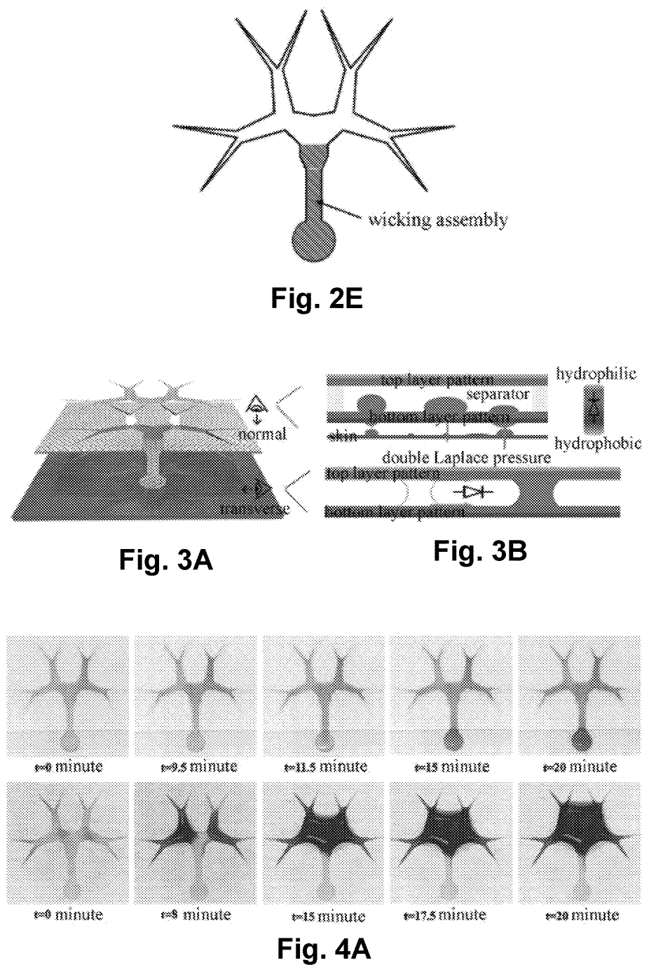

In order to prepare a mask solution, a mixed solution is prepared from 13 g of polyvinyl alcohol (PVA) and 100 ml of deionized water, and then subjected to continuous magnetic stirring at a temperature of 90° C. for 8 hours. Then, the fabric with the aluminum oxide attached to the surface (fabric-Al₂O₃) is attached to a screen-printed frame having, for example, a branch pattern (see, for example, the branch pattern shown in FIG. 2A to 2E). The prepared PVA solution is distributed evenly on the fabric, preferably using a scraper to ensure even application of the PVA solution. After removal of the screen-printed frame, the fabric is dried at the ambient temperature to facilitate curing of a PVA layer.

In some embodiments, for the top-layer microfluidic pattern, the PVA coated fabric is placed rapidly in the oven at 80° C. to accelerate curing, thereby minimizing PVA permeation into the fabric. A hydrophobic solution is prepared by adding 4 g of Nuva-N1811 into every 100 mL of distilled water to obtain an Nuva-N1811 solution and magnetically stirring the Nuva-N1811 solution for 1 hour, and then, the mask fabric is immersed in the hydrophobic solution for 10 minutes, taken out and then put in the oven at 150° C. to be cured for 20 minutes. Subsequently, the treated fabric is washed through boiling water to remove the PVA layer, and then dried in the oven at 60° C.

In some embodiments, in order to make a double-layer microfluidic pattern, a hot-melt web is used as the separation layer. The hot-melt web is precisely cut into a grid conforming to a predetermined pattern using a laser technology. The predetermined pattern of the hot-melt web is consistent with the pattern of the top layer as shown in FIG. 2D, but has a hollow design, an intermediate pattern is removed (that is, the pattern shown in FIG. 2D is removed), and a peripheral frame is reserved. Since the grid has a certain thickness, a gap can be formed between the upper layer pattern and the lower layer pattern as a sweat collection flow channel. The top (upper) layer fabric, the bottom (lower) layer fabric and the hot-melt separation layer are bonded using a 100° C. hot press for 3 minutes. Thus, one gap is formed between the top layer pattern and the bottom layer pattern, and a height of the gap corresponds to a thickness of the separation layer.

The top layer fabric and the bottom layer fabric of the fully passive mode microfluidic system are hydrophobic as a whole, but the pattern part (see, for example, the branch pattern shown in FIG. 2A to 2E) is hydrophilic, so that water in the microfluidic system is easily concentrated to the pattern part.

### Design of Sweat Collection Region and Sensing Region of Fully Passive Mode Microfluidic System

As shown above, substrates of the fabrics forming the top layer and the bottom layer of the microfluidic system are subjected to the treatment process to achieve overall hydrophobicity. Then, local branch water channels are further formed in the substrates of the top layer and the bottom layer of the microfluidic system. The local water channels have hydrophilicity, and the rest of the top layer and the rest of the bottom layer have hydrophobicity. FIG. 2A is a schematic plan diagram of the bottom layer of the microfluidic system according to some embodiments of the present invention, including the sweat collection region (indicated by a large rectangular box) and the sensing region (indicated by a small rectangular box). FIG. 2B is a schematic plan diagram of the top layer of the microfluidic system according to some embodiments of the present invention, including only the sweat collection region.

As shown in FIGS. 2A and 2B, the sweat collection region includes branched water channels that are shaped like branches and are organized under a tree-like, multi-stage arrangement. In particular, the branched water channels located at end points in this tree-like, multi-stage arrangement form a wedge shape in a transverse direction, and the other channels in fluid communication with each other are in a trapezoidal geometric shape. In order to bond the top and bottom layers while establishing an air gap between two layers of channels, a fabric-based separator (the first separation layer as shown in FIG. 1) is used, and the fabric-based separator is a hot-melt bonding grid with a fabric substrate, has a bonding function under heat treatment, and can bond the top layer fabric and the bottom layer fabric together. Since the grid has a certain thickness, one gap can be formed between the upper layer pattern and the lower layer pattern as the sweat collection flow channel. The design is characterized by a smaller inlet width and is systematically widened towards an outlet (L′1<L1 and L′0<L0). In addition, the local branch water channels are identical at the bottom and top layers (FIG. 2A to 2E).

In the branched water channels, two or more channels branched from one water channel are considered as next-stage water channels. Although the embodiment shown in FIG. 2A to 2E shows only three stages of water channels, the present invention is not limited thereto; in other embodiments, more stages of water channels may be used, or only two stages of water channels may be used as needed.

In order to collect the sweat via the end branch channels, a capillary valve is provided between the collection region and the sensing region shown in FIG. 2A. In order to facilitate the transport of the sweat from the end branch channels to the sensing region, a capillary valve having a divergence angle α is provided between the collection region and the sensing region of the bottom layer, where α ranges from 0 degrees to 90 degrees. The sensing yarn is arranged on the sensing region of the bottom layer. For example, the sensing yarn is fixed on the sensing region in an embroidering mode, and the sensing yarn is used for monitoring a biomarker in the sweat in real time.

The microfluidic system according to the embodiments of the present invention typically has a size of 16 square centimeters. The thickness of the separation layer ranges from 0.1 mm to 1 mm. However, the present invention is not limited thereto.

In addition, a wicking assembly (FIG. 2E) may be integrated into a region between the collection region and the sensing region to rapidly transport the sweat. As shown in FIG. 2E, the wicking assembly includes two parts: a cylindrical transport part and a circular collection part. A size of the cylindrical transport part should not exceed that of an original pattern.

### Sweat Transport Process and Result in Fully Passive Mode Microfluidic System

Initially, the sweat is directionally transported from the skin to the bottom layer by a porous moisture gradient across the pattern in a perpendicular direction (FIGS. 3A and 3B). This humidity gradient is created by PVA screen printing and hydrophobic modification and is controlled by mobility of the PVA solution at different temperatures. The humidity gradient is established by PVA screen printing and subsequent hydrophobic modification. This gradient is controlled by manipulating the mobility of the PVA solution at different temperatures. A high temperature enhances the mobility of the PVA, thus enabling it to vertically permeate into the fabric and wrap a fiber surface during curing. The bottom layer pattern is made using the PVA at a temperature of 80° C., and the PVA at the ambient temperature is applied to the top layer pattern. This process maintains inherent hydrophilicity of the PVA even after hydrophobic treatment. Thus, the vertical humidity gradient is generated, and is characterized in that an amount of the PVA gradually decreases from a screen printing side to an opposite side. Subsequently, the sweat is continuously absorbed into an interior of the bottom pattern by an inherent capillary action of the porous medium until saturation for absorption is reached. After saturation, the absorbed sweat is discharged to a surface of the bottom layer to form a droplet. Then, this droplet expands gradually until it reaches the top layer. Third, the sweat is transported to the sensing region under the action of a Laplace pressure from the top and bottom layers. Incorporation of the wedge geometric shape into the end branch pattern realizes the in-plane transport (i.e. transverse transport) of the sweat, ultimately resulting in sweat accumulation at the sensing region.

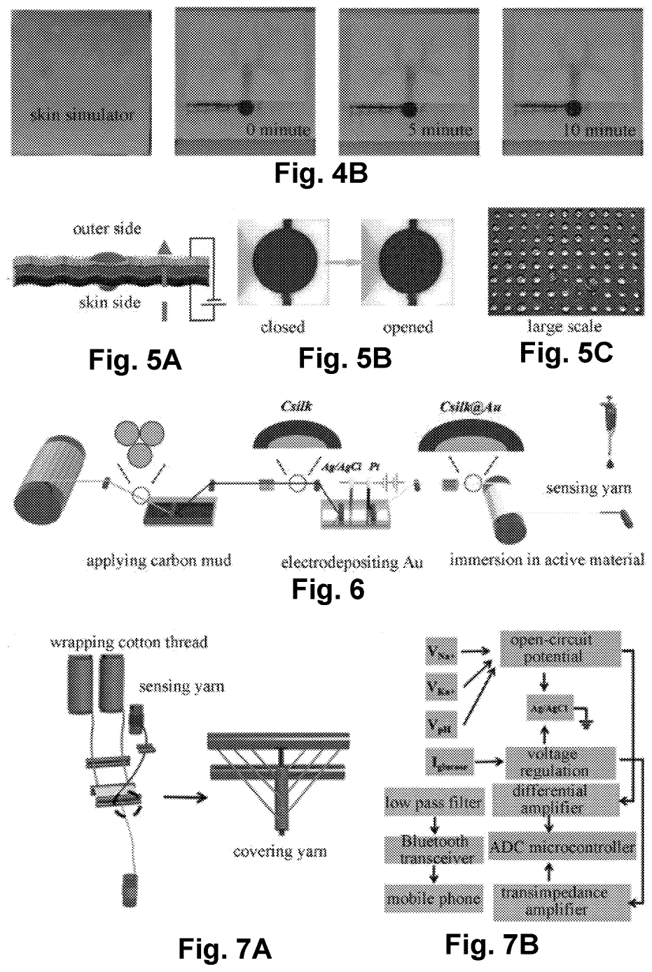

As shown in FIG. 4A, images depicting sweat flowing modes within all tree-like branches of the structure of the passively induced microfluidic system are captured without the sensing electrode when a sweating simulation system of a skin sweating simulator continuously supplies the sweat at a flow rate of 2 μL/min/cm², and a single layer is selected as a control sample. The upper row of FIG. 4A shows photographs of the double-layer microfluidic system according to certain embodiments of the present invention. In contrast, the bottom row of FIG. 4A shows photographs of a single-layer microfluidic system.

In the photographs of the upper row of FIG. 4A, i.e. the photographs of the double-layer microfluidic system according to certain embodiments of the present invention, it is observed that the microfluidic system is completely saturated with water within 11 minutes and the liquid proceeds towards the sensing region. Subsequently, the water begins to accumulate to form a hemisphere, and reaches a maximum volume at approximately 20 minutes.

In contrast, in the case of a single bottom layer, referring to several photographs of FIG. 4B, the water is directionally transported to end regions of the third-stage branches, and this process benefits from the geometric shape of the water channel structure. In the three photographs at 0, 5 and 10 minutes in FIG. 4B, the droplet is gradually formed in the collection region. It is observed during an actual test that at 15 minutes, the water spills out of the channel, rather than continuing to flow directionally to the sensing region. This phenomenon attributes to a reduction of the Laplace pressure (i.e. capillary force) which is caused by the fact that an area of the tree-like branched water channel is larger than that of the capillary valve, and the pressure is not enough to push the water towards the sensing region. About 140 microliters of sweat may be obtained in 20 minutes, but the sweat does not reach the sensing region.

### 2. Active-Passive Mode Microfluidic System

### Preparation of Active-Passive Mode Microfluidic System

For the active-passive combined mode, the electroosmotic flow apparatus facilitates the normal transport of the sweat, and the mechanism of the in-plane transport of the sweat is the same as that of the fully passive mode.

In some embodiments, the fabric-based electroosmotic flow apparatus is formed by sandwiching a microporous nylon membrane and a carbon fabric using porous adhesive liners. In some other embodiments, some dashed-line-shaped regions of an outer surface of the fabric-based electroosmotic flow apparatus are mainly subjected to hydrophobic treatment and hydrophilic treatment. Such a pattern may also be rectangular, triangular, etc. The sweat can be controllably transported normally based on electroosmosis, and the shown geometric shape is applied to sweat in-plane transport. The detailed preparation process is as follows.

According to some embodiments of the present invention, a scalable screen printing method is applied to produce an oversized fabric with a wettability pattern, and the fabric can be cut into several small-sized samples for use in an electric-wicking apparatus. A carbon fabric is first washed with distilled water and dried in an oven. A hydrophilic coating solution is prepared by adding 2 g of Hansi WS in 100 ml of distilled water. Afterwards, the fabric is soaked in the solution and cured in a heating oven at 180° C. for 2 minutes. Here, the hydrophilic fabric may be directly used as an inner layer of the electrode. Then, a mask having a hollow dot pattern is produced for screen printing. Polyvinyl alcohol (PVA) is added into water at a solution concentration of 15%, and the solution is magnetically stirred at 90° C. for 8 hours to prepare a mask solution. The fabric is fixed on a designed screen mask and the PVA solution is poured onto the mask. The mask solution is smoothed with a scraper. Thereafter, the mask is removed from the fabric, and the fabric is heated, for example, in the oven at 135° C. for 10 minutes. Next, a hydrophobic coating solution is prepared. For example, the hydrophobic coating solution is obtained by adding 4 g of Novec N1811 in 100 ml of distilled water and magnetically stirring the solution for 1 hour. The fabric covered with the PVA mask is immersed into the hydrophobic coating solution and cured in the oven at 150° C. for 2 minutes. The coating sample is immersed in boiling water until all PVA is dissolved and washed away. Finally, the sample is dried in the oven at 150° C. for 3 minutes. In a preferred embodiment, a polyester fabric may be subjected to patterned wettability treatment and attached to the outer carbon fabric, so that an outer surface thereof has the same appearance and hand feeling as a surrounding region.

According to some embodiments, the device incorporates a portable power and control unit. Two electrodes are connected to a power box (including three miniature button cells) by thin conductive yarns. A tiny vent valve is used to control an applied voltage according to different levels of sweating or personal feelings. Here, a sweat dissipation rate is adjusted in 3 levels (i.e. low, medium and high). The voltage and power consumption are predetermined according to theoretical calculations to achieve appropriate liquid dissipation rates at different levels. A tiny 3D printed TPU shell is made to store the button cell and embedded into a hollow band on sportswear. The conductive yarns are embedded and stitched within the band. A small clip can be used to securely fix the box to a waistband of a wearer. A total weight of the power and control unit is less than 20 g. The passive mode microfluidic system that can transport the sweat in a plane is fixed to the above-described device by an adhesive liner. This layer is the same as that in the fully passive mode microfluidic system.

### Testing of Sweat Transport Effect of Electroosmotic Flow Apparatus

FIG. 5A shows an illustration of electroosmosis inducing the normal transport. Different sweat dissipation rates based on certain sweat glands are measured and recorded at different voltages. It should be noted that sweat transport rates exceed 100 g/h when the voltage is 9V, which indicates that if a number of the sweat glands in the electroosmotic flow apparatus is increased to 50, a sufficient flow rate can be provided for a high sweating state of the wearer to rapidly transport the sweat secreted by the wearer away from the skin. Three conventional coin cells may last for 14.6 hours for 10 sweat glands and about 3 hours for 50 sweat glands. The time is usually sufficient because the wearer cannot be in an extremely high sweating state for a long time (e.g., over 3 hours).

The dissipation rate can be adjusted according to different applied voltages. It should be noted that the liquid rapidly dissipating in the form of the droplet on the surface of the carbon or polyester fabric is quite pronounced (FIG. 5B), which indicates that the sweat may be transported normally. Meanwhile, the large-scale electroosmotic flow apparatus is implemented to uniformly transport the sweat by increasing the voltage (FIG. 5C).

### 3. Electrochemical Sensing Yarn

### Preparation of Sensing Yarn

FIG. 6 schematically shows a process for preparing an electrochemical sensing yarn according to some embodiments of the present invention. Referring to FIG. 6, three degummed filaments are twisted into a spiral shape and then pass through a carbon paint to coat surfaces of the filaments with the carbon paint. Subsequently, the carbon paint-coated yarn is cured at 80° C. for 1 hour. This process is repeated three times to obtain a conductive yarn that is called C silk (Csilk). The C silk is used directly as a counter electrode (CE). A reference electrode (RE) is prepared by further coating the dried C silk with Ag/AgCl ink. The C silk is immersed in a 0.1% HAuCl₄ solution containing 0.5M Na₂SO₄ for electrodeposition. This process is performed using cyclic voltammetry (CV), and an external platinum counter electrode and an Ag/AgCl reference electrode (RE) are used to perform scanning at a scanning rate of 50 m V/s for 50 cycles from −1.5V to 1.5V. After the electrodeposition process, a surface of the electrode is thoroughly rinsed with ultrapure water to eliminate any residual HAuCl₄ solution. The obtained yarn is called C-silk@Au and used as a working electrode (WE).

In a preferred embodiment, a glucose sensing fiber is prepared by electrodepositing polyaniline and platinum nanoparticles as intermediates to increase sensitivity of the sensing fiber. The sensing fiber is coated with glucose oxidase fixed in a CNT/chitosan composite matrix as an active layer. An ion sensing fiber is prepared by electrodepositing PEDOT: PSS as an ion-electron conversion layer and performing drop coating on selective ion carriers as an ion specific adsorption layer. Since a surface protonation change of polyaniline at different pH values shows a large potential change, the polyaniline can be preferably used as a sensor to obtain a pH sensing optical fiber. An Na+ selective membrane contains a mixture of sodium tetraphenylborate (NaTFPB, 1.1 mg), high molecular weight polyvinyl chloride (PVC, 66 mg), bis(2-ethylhexyl) sebacate (DOS, 130 mg), and ion carrier X sodium (1 mg), 200 mg of the mixture is dissolved in 1320 μL of tetrahydrofuran, and the product is stirred for 2 hours to obtain the Na+ selective membrane. Likewise, a K+ selective membrane is prepared by dissolving sodium tetraphenylborate (NaTPB, 1 mg), PVC (65.5 mg), DOS (129 mg), and valinomycin (4 mg) in 400 μL of cyclohexanone. In order to minimize a potential drift of the ion-selective electrode, the PEDOT: PSS is used as an ion-electron transducer and deposited onto the electrode by galvanostatic electrochemical polymerization from a solution containing 0.02M of EDOT and 0.2M of NaPSS using an external Ag/AgCl reference electrode, and the unit M indicates mol/L, i.e. mole per liter. Next, the ion-selective membrane is prepared by placing 8 μL of Na+ selective membrane precursor solutions and 8 μL of K+ selective membrane precursors on the corresponding electrodes.

In some embodiments, the shell is made of fabric cotton fibers, and the sensing yarn is wrapped and protected in a protective layer (FIG. 7A). A covering yarn is manufactured using a ring spinner with additional tension and filament guiding apparatuses. A manufacturing process of the covering yarn can be simply described as follows. The prepared sensing yarn passes through a tension apparatus and a yarn guide, and is stretched by a certain pulling force, and a position of the sensing yarn is adjusted in the middle of the covering yarn to form a core of the covering yarn. A cotton thick yarn is fed into a drafting zone of the ring spinner, and a final drafted fiber exits a front roller and is wound onto the sensing yarn to form the protective surface layer. All the prepared yarn electrodes are embroidered on the sensing region of the microfluidic system and at the same time connected to the flexible PCB for wireless signal transmission.

### Sensing Characteristics of Each Electrode

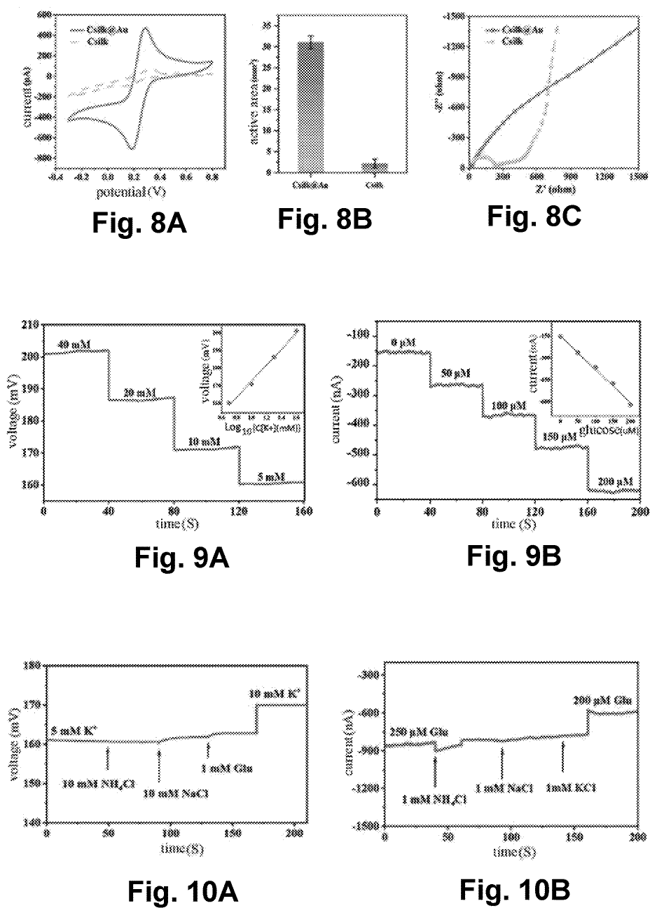

FIG. 8A shows a graph of cyclic voltammetry measurement using a ferricyanide redox probe; FIG. 8B is a bar graph of active surface areas of two yarn electrodes. Here, the electrochemically active surface area of the electrode is estimated by cyclic voltammetry measurement using the ferricyanide redox probe. A measurement result conforms to a model derived from the following Randles-Sevcik equation, and the model characterizes a reversible redox system.

$${i}_{p} = ( 2.69 \times 1 {0}^{5} ) {n}^{3 / 2} {ACD}^{1 / 2} {v}^{1 / 2}$$

wherein iₚ is a peak current obtained from cyclic voltammetry in a ferricyanide solution, n is a number of electrons transferred in a redox event, A is the electrochemically active surface region, C is a concentration, D is a diffusion coefficient, and v is the scanning rate. As shown in FIG. 8B, the electrochemically active surface area of the yarn electrode is increased significantly from 2.2 mm² to 31.1 mm² after Au particles are deposited on a surface of the electrode. Subsequently, a charge transfer resistance (Rct) is determined using electrochemical impedance spectroscopy. The result shown in FIG. 8C indicates that C-silk@Au is about 9.2 ohms and is significantly lower than 206.5 ohms for the original C silk. The significant reduction of Rct highlights efficacy of the deposited Au nanoparticles in promoting an electron transfer efficiency.

FIGS. 9A and 9B show calibration graphs of the K+ and glucose sensing electrodes respectively. A potentiometric ion-selective electrode is adopted for continuous K+ monitoring, and in the electrode, an interaction of potassium with its ion carriers leads to an electrode potential (FIG. 9A). A thin coating of polyvinyl butyral (PVB) is applied to the reference electrode and is used for stabilizing the voltage to prevent changes inherent in sweat biofluids. The prepared K+ electrode and the concentration of target ions are in a logarithmic linear relationship, and sensitivity of 45.3 mV per decade is achieved. Precise and uninterrupted quantification of glucose is realized using an amperometric enzyme electrode. The electrode incorporates glucose oxidase fixed in a chitosan film, and is characterized by high permeability, adhesiveness and biocompatibility. Addition of electrodeposited Prussian Blue (PB) as an electron transfer redox medium facilitates an enzymatic reaction. FIG. 9B shows a corresponding calibration curve of the glucose electrode in the range of 0-200 μM. A resulting calibration relationship exhibits good linearity (R2=0.99) and sensitivity (2.3 nA/μM).

Selectivity of the electrochemical sensor is of critical importance because various biomarkers present in the sweat may interfere with detection of the target biomarker. In order to assess this point, interfering ions are purposefully mixed into a targeted biomarker solution to facilitate observation of current or potential subsequent changes. As shown in FIGS. 10A and 10B, a reaction of adding the interfering ions or metabolites is less pronounced compared to the target biomarker, which indicates that the prepared electrode has excellent selectivity.

### 4. Integration of Sweat Sensor System

### Design and Manufacture

The sweat sensor is integrated with a signal collection and transmission circuit assembly for wireless, in-vivo and real-time sweat analysis. The integrated system comprises different functional parts including signal transduction, conditioning, processing and wireless transmission from the sensor to a mobile phone. In order to verify the concept, the fabric sweat sensor is located on an upper right side of a back of the sportswear or T-shirt (slightly below a right shoulder) for real-time sweat analysis. A sensing chip is detachable, and the sensing yarn can be quickly connected and inserted through a conductive yarn connector. The chip is placed in a small pocket sealed at the shoulder. The ergonomic design ensures that the sweat collection region is always in contact with the skin of the upper back, which has a high sweating rate according to a body sweat map. The biomarker in the sweat from a human subject (during physical activity) is tracked in real time and the result can be displayed on a smartphone for non-invasive health monitoring.

The sensing chip mainly includes an ultra-low power consumption mixed signal microcontroller (Aducm355 SoC device), an FT232 USB interface IC and a small Bluetooth low energy module (RN4871 BLE module). As a core of the system, the microcontroller is programmed on a board through an on-line serial programming interface. The system further includes an analog signal conditioning path that terminates in a unity gain four-pole low pass filter. This filter is configured to process a current of a glucose channel and voltages at sodium and potassium channel terminals. An active filter is configured to fine adjust a gain in the signal conditioning path. These low pass filters are connected to an analog-to-digital converter stage of the microcontroller, and convert filtered analog signals into respective digital formats. By utilizing a built-in 10-bit analog-to-digital converter block of the microcontroller and its computation and serial communication capabilities, the signal (converted by the sensor module of the present invention and conditioned by the analog circuit of the present invention) is relayed to a Bluetooth transceiver. The sensing signal is then displayed on a mobile device in real time (FIG. 10B).

### Performance

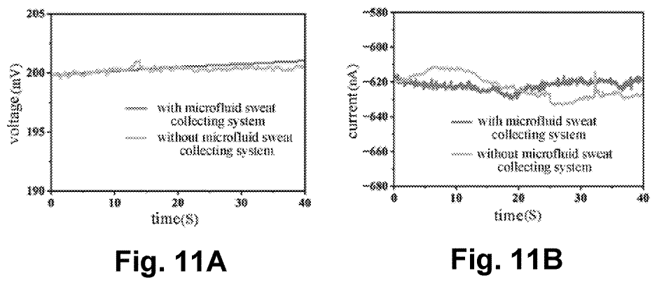

In order to further verify signal stability within the microfluidic system, 40 mM of K+ solutions are applied to the yarn electrodes integrated inside and outside the full-fabric microfluidic system. It can be observed that in case of sweat microfluidic collection according to the present invention, the output signal remains stable within the microfluidic system, whereas fluctuations exist without the output signal (FIG. 11A). In contrast, similar results are obtained for the glucose sensing electrode without microfluidic sweat collection according to the present invention (FIG. 11B). These findings indicate that the microfluidic system according to the present invention is not only effective in sweat collection, but also maintains the signal stability, thereby improving monitoring precision.

While the invention is described with reference to specific embodiments, it will be understood by those skilled in the art that various changes may be made and equivalents may be substituted without departing from the scope of the invention. In addition, many modifications may be made to the invention for a particular situation or material without departing from the scope of the invention. Therefore, the invention is not limited to the particular embodiment disclosed, but should include all embodiments falling within the scope of the appended claims.

What is claimed is:

1. A microfluidic sweat collecting and sensing system, the system comprising:
a bottom layer arranged to be positioned on a skin of a wearer;
a top layer; and
a separation layer located between the bottom layer and the top layer;
wherein the separation layer is made of a hot-melt material;
wherein the top layer, the bottom layer and the separation layer are bonded together by hot pressing, so that a gap is formed between the top layer and the bottom layer as a sweat collection flow channel; and
wherein each of the top and bottom layers is formed with a plurality of water channels.

2. The microfluidic sweat collecting and sensing system according to claim 1, wherein the bottom layer comprises a first collection region and a sensing region, and wherein the top layer comprises a second collection region.

3. The microfluidic sweat collecting and sensing system according to claim 2, wherein in each of the first and second collection regions, the plurality of water channels includes branched water channels organized with a tree-like, multi-stage branching arrangement, wherein a first branched water channel selected from the branched water channels and located at an end point has a wedge shape with a tip pointing outwards, wherein a second branched water channel selected from the branched water channels and connected to the first branched water channel has a shape of a trapezoid, and wherein a width of the trapezoid is small at an inflow end of a water stream and is larger at an outflow end of the water stream.

4. The microfluidic sweat collecting and sensing system according to claim 3, wherein a capillary valve having a divergence angle α is arranged between the first collection region and the sensing region of the bottom layer to facilitate transport of sweat from the first collection region to the sensing region, and α ranges from 0 degrees to 90 degrees.

5. The microfluidic sweat collecting and sensing system according to claim 2, wherein a sensing yarn is arranged on the sensing region of the bottom layer for real-time monitoring of biomarkers in the sweat.

6. The microfluidic sweat collecting and sensing system according to claim 2, further comprising a wicking assembly integrated into a region between the first collection region and the sensing region to facilitate sweat transport, wherein the wicking assembly comprises a cylindrical transport part and a circular collection part.

7. The microfluidic sweat collecting and sensing system according to claim 1, wherein the separation layer has a thickness ranging from 0.1 mm to 1 mm, and the microfluidic sweat collecting and sensing system has an area of 16 cm².

8. The microfluidic sweat collecting and sensing system according to claim 1, further comprising an electroosmotic flow apparatus secured to the bottom layer and configured to facilitate normal transport of the sweat.

9. The microfluidic sweat collecting and sensing system according to claim 8, wherein the electroosmotic flow apparatus is formed by sandwiching a microporous nylon membrane and a carbon fabric using porous adhesive liners.

10. The microfluidic sweat collecting and sensing system according to claim 5, wherein the sensing yarn is wrapped with a skin-friendly fiber.

11. The microfluidic sweat collecting and sensing system according to claim 5, wherein an active layer of the sensing yarn comprises glucose oxidase and an ion-selective membrane.

12. The microfluidic sweat collecting and sensing system according to claim 1, wherein fabrics forming the bottom layer and the top layer are hydrophilic in parts corresponding to the water channels and hydrophobic in remaining parts.
